(19) United States
(12) Patent Application Publication
(10) Pub. No.: US 2024/0037387 A1
(43) Pub. Date: Feb. 1, 2024

(54) POWER TRANSFORMER FAULT DIAGNOSIS METHOD BASED ON STACKED TIME SERIES NETWORK

(71) Applicants: WUHAN UNIVERSITY, Wuhan (CN); State Grid Tianjin Electric Power Company, Tianjin (CN)

(72) Inventors: Yigang HE, Wuhan (CN); Zhikai XING, Wuhan (CN); Xiao WANG, Wuhan (CN); Xiaoyu LIU, Wuhan (CN); Xue JIANG, Wuhan (CN); Qingwu GONG, Wuhan (CN); Jianfeng WANG, Wuhan (CN); Shiqian MA, Wuhan (CN)

(73) Assignees: WUHAN UNIVERSITY, Wuhan (CN); State Grid Tianjin Electric Power Company, Tianjin (CN)

(21) Appl. No.: 18/060,583

(22) Filed: Dec. 1, 2022

(30) Foreign Application Priority Data
Jul. 26, 2022 (CN) 202210884861.5

(51) Int. Cl. G06N 3/08; G05B 23/02

(52) U.S. Cl. CPC G06N 3/08; G05B 23/0283

(57) ABSTRACT

A power transformer fault diagnosis method based on a stacked time series network, includes: collecting gas-in-oil data of a transformer in each substation; performing z-score normalization on the collected data to obtain a normalized matrix; dividing the normalized matrix into a training set and a test set in proportion; constructing a stacked time series network based on Xgboost and a bidirectional gated neural network, and inputting the training set and the test set to perform network training; and normalizing real-time collected data to obtain trainable data to predict a fault and update network parameters. The gas-in-oil data is predicted by using Xgboost and a gated neural network, obtains prediction data of a power transformer from two time series networks by using a meta learner, and obtains a fault diagnosis result of the transformer by using a Softmax layer. The neural network has accurate fault diagnosis performance and stable robustness.

### CROSS-REFERENCE TO RELATED APPLICATIONS

This application claims priority to Chinese Patent Application No. 202210884861.5 with a filing date of Jul. 26, 2022. The content of the aforementioned application, including any intervening amendments thereto, is incorporated herein by reference.

### TECHNICAL FIELD

The present disclosure relates to the technical field of fault diagnosis of power transformers, and more specifically, to a power transformer fault diagnosis method based on a stacked time series network.

### BACKGROUND

As one of most important electrical devices in a power system, a high-voltage large-capacity transformer has a complex structure and is expensive. It plays an important role in safe and stable operation of the power system. Once the transformer is faulty, a huge economic loss is caused. As a very important voltage conversion node and a core device of a substation, a power transformer is expensive. Sudden shutdown of the power transformer due to a fault will seriously endanger security of the power system. However, longstanding preventive testing and regular maintenance cannot meet an operation and maintenance requirement of the high-voltage large-capacity transformer. How to supply power safely without affecting people's quality of life and work efficiency is a huge challenge faced by power workers in China.

Power transformer fault diagnosis is an effective means to determine a fault state of the transformer. Data accumulated during operation of the transformer contains rich state information, which is an effective basis for state evaluation and fault diagnosis. With the overall construction and promotion of a smart grid, multi-source and heterogeneous power big data increases explosively. Traditional gas-in-oil fault diagnosis methods cannot meet development needs of power enterprises. Existing fault diagnosis methods perform fault diagnosis based on current temporal data, but there is a trend of change when a fault occurs on the transformer. A new fault diagnosis technology based on time series data still needs to be studied. However, in an actual time series prediction task, due to a similarity between modal data, the time series data is redundant to a certain degree. If a single time series model is used, performance of the model in a target task is often reduced.

### SUMMARY

To overcome the defects or meet the improvement demands in the prior art, the present disclosure provides a power transformer fault diagnosis method based on a stacked time series network, to revolve a problem that the prior art relies too much on selection of variable parameters and cannot diagnose a fault state at a future time point. The method is characterized by simple operations and high diagnosis accuracy, making it easy to diagnose a transformer fault at the future time point.

To achieve the above objective, the present disclosure provides a power transformer fault diagnosis method based on a stacked time series network, including:

(1) collecting gas-in-oil information of each substation, where the gas-in-oil information includes monitoring information of an oil test and contents of dissolved gas and furan in oil in each substation;

(2) normalizing the collected gas-in-oil information to obtain a normalized matrix;

(3) dividing the normalized matrix into a training set and a test set in proportion to train network parameters;

(4) constructing a stacked time series network based on Xgboost and a bidirectional gated neural network, inputting the training set and the test set to train the stacked time series network, and learning a feature of gas-in-oil data of a transformer; and

(5) performing fault diagnosis based on real-time gas-in-oil data during operation, and fine tuning a weight of the stacked time series network to enable the stacked time series network to continuously learn a new feature.

In some optional implementation solutions, the gas-in-oil information includes data of the transformer during operation and data recorded by an electric power company, and each group of data includes gas-in-oil data and a fault state of the corresponding transformer, where the gas-in-oil data includes contents of nine key states: a breakdown voltage (BDV), water, acidity, hydrogen, methane, ethane, ethylene, acetylene, and furan.

In some optional implementation solutions, step (2) includes: performing z-score normalization on the gas-in-oil information to obtain the normalized matrix.

In some optional implementation solutions, the data in the gas-in-oil information is divided into two parts in step (3), where data of a certain proportion is used as the training set to train the stacked time series network, and a data of a remaining proportion is used as the test set to test a fault diagnosis effect of the stacked time series network for the transformer.

In some optional implementation solutions, step (4) includes:

(4.1) constructing the stacked time series network based on Xgboost and the bidirectional gated neural network to perform feature extraction and prediction on the gas-in-oil information, where construction of Xgboost includes establishment of an integrated model, selection of an objective function, and solving of a loss function; and the bidirectional gated neural network includes a forward calculation layer, a backward calculation layer, an update gate, and a reset gate;

(4.2) predicting gas in the oil by using Xgboost and the bidirectional gated neural network, and outputting a prediction result of the gas-in-oil information; and

(4.3) training prediction results of Xgboost and the bidirectional gated neural network by using a meta learner, to output the prediction result of the gas-in-oil information, performing fault diagnosis on the stacked time series network by using a Softmax layer, and outputting the fault state of the transformer.

In some optional implementation solutions, the construction of Xgboost includes the establishment of the integrated model, the selection of the objective function, and the solving of the loss function, where the establishment of the integrated model is to recursively construct a binary decision tree, and in input space of the training set, each region is recursively divided into two sub-regions based on a minimum squared-error criterion, and an output value of each sub-region is determined; the selection of the objective function is to measure an error between a predicted value and a real value of a target, and the objective function is approximated through second-order Taylor expansion; and the solving of the loss function is to partition a sub-tree by using a greedy algorithm, enumerate feasible partitioning points, in other words, add a new partition to an existing leaf each time, and calculate a corresponding maximum gain.

In some optional implementation solutions, the bidirectional gated neural network includes the forward calculation layer, the backward calculation layer, the update gate, and the reset gate, where the reset gate helps to capture a short-term dependency in a time series, the update gate helps to capture a long-term dependency in the time series, and the forward calculation layer and the backward calculation layer process the input series in turn.

In some optional implementation solutions, the meta learner trains and predicts the results of Xgboost and the bidirectional gated neural network, and the meta learner is constructed as a linear regression model to learn and predict the results of Xgboost and the bidirectional gated neural network.

In some optional implementation solutions, step (5) includes:

performing z-score normalization on real-time collected gas-in-oil data, and then dividing normalized data into the training set and the test set to train the stacked time series network for fault diagnosis, where if a new data type or a relevant influencing factor needs to be added, the original stacked time series network is taken as a pre-training model to activate all layers for training.

Compared with the prior art, the above technical solutions conceived by the present disclosure can achieve the following beneficial effects:

Considering that no time series is considered and accuracy is low in a typical fault diagnosis process of the transformer, the power transformer fault diagnosis method based on a stacked time series network in the present disclosure uses the stacked time series network for fault diagnosis. Considering a different prediction ability and feature extraction ability of the stacked time series network comprehensively, the present disclosure creatively constructs a fault diagnosis model of the stacked time series network based on Xgboost and the bidirectional gated neural network. On this basis, because units of actual engineering data are different, z-score normalization is performed on input data of the model.

### BRIEF DESCRIPTION OF THE DRAWINGS

FIG. 1 is a flowchart of implementing a power transformer fault diagnosis method based on a stacked time series network according to an embodiment of the present disclosure;

FIG. 2 shows an Xgboost structure according to an embodiment of the present disclosure;

FIG. 3 shows a structure of a bidirectional gated neural network according to an embodiment of the present disclosure; and

FIG. 4 shows a structure of a model based on a stacked time series network according to an embodiment of the present disclosure.

### DETAILED DESCRIPTION OF THE EMBODIMENTS

To make the objectives, technical solutions, and advantages of the present disclosure clearer, the present disclosure is further described below in detail with reference to the drawings and embodiments. Understandably, the specific embodiments described herein are merely intended to explain the present disclosure but not to limit the present disclosure. Further, the technical features involved in the various embodiments of the present disclosure described below may be combined with each other as long as they do not constitute a conflict with each other.

The present disclosure is intended to provide a new fault diagnosis method for a transformer state, to achieve higher fault diagnosis efficiency and accuracy and resolve a problem that a traditional method does not consider fault diagnosis in the case of a time series.

The present disclosure is implemented by the following technical solutions.

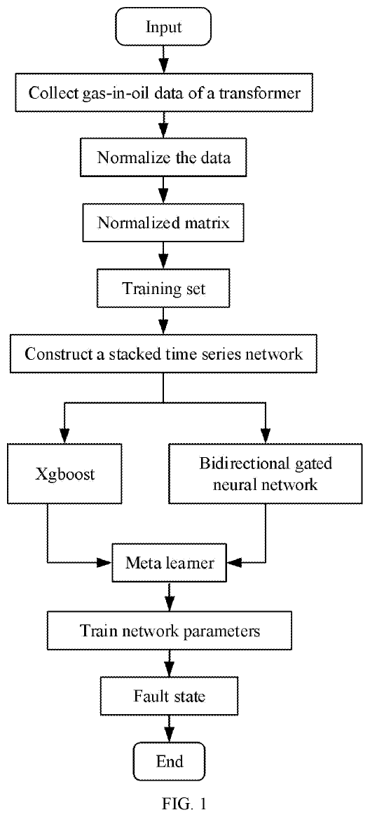

As shown in FIG. 1, a power transformer fault diagnosis method based on a stacked time series network according to an embodiment of the present disclosure includes the following steps.

Step 1: Collect monitoring information of an oil test and contents of dissolved gas and furan in oil in each substation.

The data in step 1 is collected from data of a transformer during operation and test data of an electric power company, and each group of data includes contents of nine key states: a BDV, water, acidity, hydrogen (H₂), methane (CH₄), ethane (C₂H₆), ethylene (C₂H₄), acetylene (C₂H₂), and furan, and a fault state of the corresponding transformer.

Step 2: Normalize collected gas-in-oil information to obtain a normalized matrix.

Specifically, step 2 may be implemented in the following manner:

performing z-score normalization on the gas-in-oil data according to the following formula:

$${y}_{i} = \frac{x - μ}{σ} \qquad ( 1 )$$

where μ and σ represent an average value and a variance of an original dataset respectively.

In this embodiment, the transformer has the following fault states: low-temperature overheating fault (T1), medium-temperature overheating fault (T2), high-temperature overheating fault (T3), partial discharge fault (PD), low-energy discharge fault (D1), and high-energy discharge fault (D2), as shown in Table 1.

TABLE 1
Fault states of the transformer

| Fault state | Quantity of samples |
|---|---|
| Low-temperature overheating fault (T1) | 256 |
| Medium-temperature overheating fault (T2) | 179 |
| High-temperature overheating fault (T3) | 141 |
| Partial discharge fault (PD) | 98 |
| Low-energy discharge fault (D1) | 221 |
| High-energy discharge fault (D2) | 194 |

Step 3: Divide the normalized matrix into a training set and a test set in proportion.

A dataset of the normalized matrix is divided into two parts, where 80% of the dataset is used as the training set to train a stacked time series network, and 20% of the dataset is used as the test set to test an effect of classifying the fault states by the stacked time series network.

Step 4: Construct a stacked time series network based on Xgboost and a bidirectional gated neural network, input the training set and the test set to perform network training.

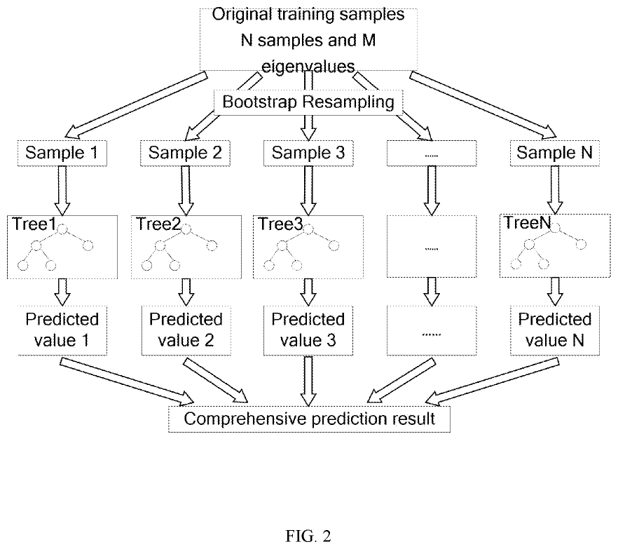

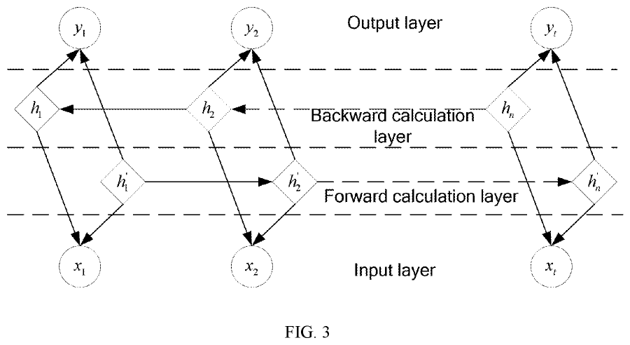

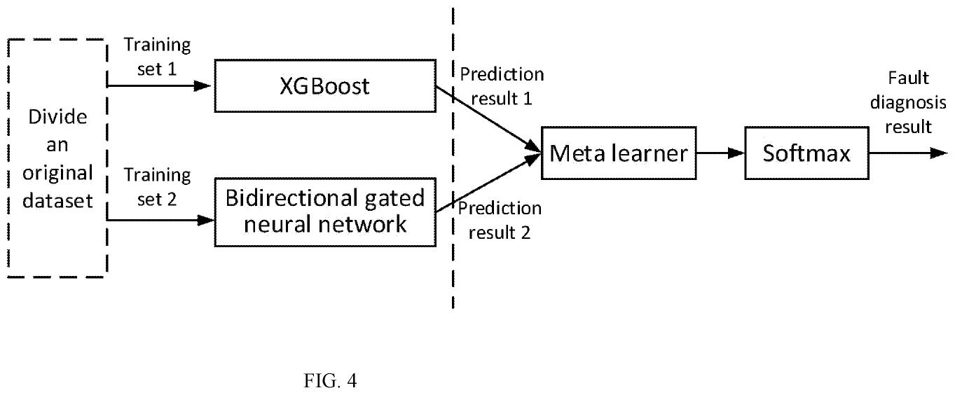

In step 4, the stacked time series network is constructed in the following manner: first, constructing an Xgboost model, as shown in FIG. 2; then constructing the bidirectional gated neural network, as shown in FIG. 3; and finally, using a meta learner to learn results of Xgboost and the bidirectional gated neural network, as shown in FIG. 4.

Specifically, step 4 may be implemented in the following manner:

Step 4.1: Construct the Xgboost model, in other words, construct a binary decision tree recursively. An integrated model of the tree is as follows:

$${\hat{y}}_{i} = {\sum }_{k = 1}^{K} {f}_{k} ( {x}_{i} ) , {f}_{k} ∈ F \qquad ( 2 )$$

where Σᵢ₌₁ⁿl(yᵢ,ŷᵢ)represents an error between a predicted value and a real value of a target, ŷᵢ represents a predicted value of the model, yᵢ represents the real value of the target, l indicates that a quantity of combined decision trees is a quantity of trees to be adjusted, fₗ represents a first tree, xᵢ represents an i^{th} input sample, F represents a set of all tree models, Σₖ₌₁^{K}Ω(fₖ) represents a regular term for controlling complexity of the model, which is used to prevent overfitting of the model, and

Ω(f)=λT+1/2·λ∥w∥².

In the above formula, T represents a quantity of leaf nodes, λ and γ represent penalty coefficients for the model, and w represents a weight of a leaf node in a tree. The model adds an incremental function fᵢ(xᵢ) in each round to minimize a loss function. An objective function of a t^{th} round can be expressed as follows:

$${L}^{( t )} = {\sum }_{i = 1}^{n} l ( {y}_{i} , {\hat{y}}_{i}^{( t - 1 )} + {f}_{i} ( {x}_{i} ) ) + Ω ( {f}_{t} ) \qquad ( 3 )$$

In order to obtain fᵢ(xᵢ) with a minimum objective function, in the above formula, second-order Taylor expansion is performed to approximate the objective function. A sample set of each leaf of a j^{th} tree is defined as

Iⱼ={i|q(xᵢ=j)}. gᵢ∂_{ŷ}_{ᵢ}_{^{(t−1)}}l(yᵢ,ŷᵢ^{(t-1)}). and

hᵢ=∂²_{ŷ}_{ᵢ}_{^{(t−1)}}l(yᵢ,ŷᵢ^{(t-1)}),

where Iⱼ and hᵢ represent a first derivative and a second derivative of the loss function respectively. Therefore, the following formula can be obtained:

$$\begin{matrix}{L}^{( t )} ≅ {\sum }_{i = 1}^{n} [ {g}_{i} {f}_{t} ( {x}_{i} ) + \frac{1}{2} {h}_{i} {f}_{t}^{2} ( {x}_{i} ) ] + Ω ( {f}_{t} ) \\ ≅ {\sum }_{i = 1}^{n} [ {g}_{i} {f}_{t} ( {x}_{i} ) + \frac{1}{2} {h}_{i} {f}_{t}^{2} ( {x}_{i} ) ] + γ T + \frac{1}{2} λ {\sum }_{j = 1}^{T} {w}_{j}^{2} \\ ≅ {\sum }_{j = 1}^{T} [ {\sum }_{i ∈ {I}_{j}} ( {g}_{i} ) {w}_{j} + \frac{1}{2} ( {\sum }_{i ∈ {I}_{j}} {h}_{i} + λ ) {w}_{j}^{2} ) ] + γ T\end{matrix} \qquad ( 4 )$$

Gⱼ=Σ_{i∈I}_{ⱼ}(gᵢ) and Hⱼ=Σ_{i∈I}_{ⱼ}hᵢ are defined, and a partial derivative is obtained for w, such that the following formula can be obtained:

$${w}_{j} = - \frac{{G}_{j}}{{H}_{j} + λ} \qquad ( 5 )$$

The weight is substituted into the objective function, such that the following formula can be obtained:

$${L}^{( t )} = - \frac{1}{2} {\sum }_{j = 1}^{T} \frac{{G}_{j}^{2}}{{H}_{j} + λ} + γ T \qquad ( 6 )$$

A smaller loss function leads to a better model. A greedy algorithm is used to partition a subtree, and feasible partitioning points are enumerated, that is, a new partition is added to an existing leaf each time. Then a corresponding maximum gain is calculated.

Step 4.2: Construct the bidirectional gated neural network.

A bidirectional gate recurrent unit (GRU) structure can process input data xᵢ=[x₁, . . . , xₙ]^{T} in both forward and backward directions, and then splice two obtained feature vectors together as another expression of an input vector.

1) A formula for forward calculation is:

\vec{z}ₜ=σ(\vec{W}_{xz}\vec{x}ₜ+\vec{W}_{hz}\vec{h}ₜ₋₁+\vec{b}_{z})

\vec{r}ₜ=σ(\vec{W}ₓᵣ\vec{x}ₜ+\vec{W}ₕᵣ\vec{h}ₜ₋₁+\vec{b}ᵣ)

\vec{g}ₜ=tan h(\vec{W}_{xg}\vec{x}ₜ+\vec{W}_{hg}(\vec{r}ₜ×\vec{h}ₜ₋₁+\vec{b}_{g})

\vec{h}ₜ=(1−\vec{z}ₜ)×\vec{h}ₜ₋₁+ [symbol] ×\vec{g}ₜ (7)

2) A formula for backward calculation is:

[symbol]

ₜ=σ( [symbol] _{xz} [symbol] ₜ+ [symbol] _{hz} [symbol] ₜ₋₁+ [symbol] )

[symbol]

ₜ=σ( [symbol] ₓᵣ [symbol] ₜ+ [symbol] ₕᵣ [symbol] ₜ₋₁+ [symbol] )

[symbol]

ₜ=tan h( [symbol] _{xg} [symbol] + [symbol] _{hg}( [symbol] × [symbol] ₜ₋₁+ [symbol] )

[symbol]

ₜ=(1− [symbol] )× [symbol] ₜ₋₁+ [symbol] × [symbol] (8)

In the above formulas, rₜ and zₜ represent an update gate and a reset gate under a current time step respectively; gₜ represents a hidden state at a current time point; hₜ₋₁ and hₜ represent states at a previous time point and the current time point respectively; W_{xz}, and Wₓᵣ, and W_{xg} represent weight matrices connected to an input vector xₜ; W_{hz}, and Wₕᵣ, and W_{hg} represent weight matrices connected to a state vector hₜ₋₁ of a previous unit; b_{z}, bᵣ, and b_{g} represent deviation vectors; σ represents an activation function, which is a sigmoid function and a gating signal of the bidirectional gated neural network; and tan h represents that the activation function scales data to [−1, 1]. Finally, \vec{h}ₜ obtained through the forward calculation and [symbol] obtained through the backward calculation are superposed as a final output of the model.

hₜ=\vec{h}ₜ+ [symbol] (9)

Step 4.3: Construct the meta learner as a linear regression model whose composition is as follows:

A new training set is formed by the prediction results of Xgboost and the bidirectional gated neural network to train a single-variable linear regression model to construct the stacked time series network. The single-variable linear regression model is:

y=mx+b (10)

A prediction result of the stacked time series network is input into a Softmax layer to obtain the fault state of the transformer.

For simplicity, the following formula is used to calculate an accuracy rate. In practical application, an output result of the softmax layer can also be comprehensively considered. Each group of data corresponds to a probability of each transformer state label. A type corresponding to a maximum probability can be selected as a diagnosis result. In addition, when there is no significant difference between the second largest probability and the maximum probability in the softmax layer, the two diagnosis results can be comprehensively considered.

$$Pc = \frac{TP}{FP + TP} \qquad ( 11 )$$

$$R = \frac{TP}{FN + TP}$$

$$F 1 = 2 \times \frac{Pc \times R}{Pc + R}$$

In the above formula, TP represents a quantity of positive categories that are predicted as positive categories, FP represents a quantity of negative categories that are predicted as positive categories, and FN represents a quantity of positive categories that are predicted as negative categories.

Step 5: Perform fault diagnosis and fine tune the neural network based on actual test data. A dataset, obtained through monitoring, of the transformer is input for fault diagnosis. A final diagnosis accuracy rate is 98.87%.

A method for performing fault diagnosis and fine tuning the neural network based on the actual test data in step 5 includes: normalizing real-time collected gas-in-oil data according to step 2, and then dividing normalized data into the training set and the test set according to step 3 to train the stacked time series network; and finally, performing fault diagnosis based on the test set in step 5, where if a new data type or a relevant influencing factor needs to be added, the original stacked time series network is taken as a pre-training model to activate all layers for training.

The present disclosure collects gas-in-oil data of a transformer in each substation; normalizes collected monitoring information to obtain a normalized matrix; dividing the normalized matrix into a training set and a test set in proportion; constructing a stacked time series network based on Xgboost and a bidirectional gated neural network, and inputting the training set and the test set to perform network training; and normalizing real-time collected data to obtain trainable data to predict a fault and update network parameters. Considering that there is gas-in-oil data in data of a power transformer, and fault diagnosis accuracy based on the gas-in-oil data is low, the present disclosure uses Xgboost and the bidirectional gated neural network to predict the gas-in-oil data, obtains a prediction result of the gas-in-oil data by using a meta learner, and obtains a fault state of the transformer by using a Softmax layer. The neural network has accurate fault diagnosis performance and stable robustness.

It should be pointed out that, based on needs of implementation, each step/component described in the present disclosure can be divided into more steps/components, or two or more steps/components or some operations of the steps/components can be combined into a new step/component to achieve the objective of the present disclosure.

It is easy for those skilled in the art to understand that the above-mentioned contents are merely the preferred embodiments of the present disclosure, and are not intended to limit the present disclosure. Any modifications, equivalent substitutions and improvements made within the spirit and principles of the present disclosure should fall within the protection scope of the present disclosure.

What is claimed is:

1. A power transformer fault diagnosis method based on a stacked time series network, comprising:
(1) collecting gas-in-oil information of each substation, wherein the gas-in-oil information comprises monitoring information of an oil test and contents of dissolved gas and furan in oil in each substation;
(2) normalizing the collected gas-in-oil information to obtain a normalized matrix;
(3) dividing the normalized matrix into a training set and a test set in proportion to train network parameters;
(4) constructing a stacked time series network based on Xgboost and a bidirectional gated neural network, inputting the training set and the test set to train the stacked time series network, and learning a feature of gas-in-oil data of a transformer; and
(5) performing fault diagnosis based on real-time gas-in-oil data during operation, and fine tuning a weight of the stacked time series network to enable the stacked time series network to continuously learn a new feature.

2. The method according to claim 1, wherein the gas-in-oil information comprises data of the transformer during operation and data recorded by an electric power company, and each group of data comprises gas-in-oil data and a fault state of the corresponding transformer, wherein the gas-in-oil data comprises contents of nine key states: a breakdown voltage (BDV), water, acidity, hydrogen, methane, ethane, ethylene, acetylene, and furan.

3. The method according to claim 1, wherein step (2) comprises: performing z-score normalization on the gas-in-oil information to obtain the normalized matrix.

4. The method according to claim 1, wherein the data in the gas-in-oil information is divided into two parts in step (3), wherein data of a certain proportion is used as the training set to train the stacked time series network, and a data of a remaining proportion is used as the test set to test a fault diagnosis effect of the stacked time series network for the transformer.

5. The method according to claim 4, wherein step (4) comprises:
(4.1) constructing the stacked time series network based on Xgboost and the bidirectional gated neural network to perform feature extraction and prediction on the gas-in-oil information, wherein construction of Xgboost comprises establishment of an integrated model, selection of an objective function, and solving of a loss function;
and the bidirectional gated neural network comprises a forward calculation layer, a backward calculation layer, an update gate, and a reset gate;
(4.2) predicting gas in the oil by using Xgboost and the bidirectional gated neural network, and outputting a prediction result of the gas-in-oil information; and
(4.3) training prediction results of Xgboost and the bidirectional gated neural network by using a meta learner, to output the prediction result of the gas-in-oil information, performing fault diagnosis on the stacked time series network by using a Softmax layer, and outputting the fault state of the transformer.

6. The method according to claim 5, wherein the construction of Xgboost comprises the establishment of the integrated model, the selection of the objective function, and the solving of the loss function, wherein the establishment of the integrated model is to recursively construct a binary decision tree, and in input space of the training set, each region is recursively divided into two sub-regions based on a minimum squared-error criterion, and an output value of each sub-region is determined; the selection of the objective function is to measure an error between a predicted value and a real value of a target, and the objective function is approximated through second-order Taylor expansion; and the solving of the loss function is to partition a sub-tree by using a greedy algorithm, enumerate feasible partitioning points, in other words, add a new partition to an existing leaf each time, and calculate a corresponding maximum gain.

7. The method according to claim 6, wherein the bidirectional gated neural network comprises the forward calculation layer, the backward calculation layer, the update gate, and the reset gate, wherein the reset gate helps to capture a short-term dependency in a time series, the update gate helps to capture a long-term dependency in the time series, and the forward calculation layer and the backward calculation layer process the input series in turn.

8. The method according to claim 7, wherein the meta learner trains and predicts the results of Xgboost and the bidirectional gated neural network, and the meta learner is constructed as a linear regression model to learn and predict the results of Xgboost and the bidirectional gated neural network.

9. The method according to claim 8, wherein step (5) comprises:
performing z-score normalization on real-time collected gas-in-oil data, and then dividing normalized data into the training set and the test set to train the stacked time series network for fault diagnosis, wherein if a new data type or a relevant influencing factor needs to be added, the original stacked time series network is taken as a pre-training model to activate all layers for training.
